(19) United States
(12) Patent Application Publication
(10) Pub. No.: US 2024/0082449 A1
(43) Pub. Date: Mar. 14, 2024

(54) APPARATUS AND METHOD FOR CREATING AROMA

(71) Applicants: Firooz Rasouli, San Diego, CA (US); Rong Wang, Hinsdale, IL (US); Hamid Arastoopour, Downers Grove, IL (US); Ali K. Oskouie, Hinsdale, IL (US)

(72) Inventors: Firooz Rasouli, San Diego, CA (US); Rong Wang, Hinsdale, IL (US); Hamid Arastoopour, Downers Grove, IL (US); Ali K. Oskouie, Hinsdale, IL (US)

(73) Assignee: ILLINOIS INSTITUTE OF TECHNOLOGY, CHICAGO, IL (US)

(21) Appl. No.: 18/244,395

(22) Filed: Sep. 11, 2023

Related U.S. Application Data
(60) Provisional application No. 63/405,182, filed on Sep. 9, 2022.

(51) Int. Cl. A61L 9/12; A61L 9/04

(52) U.S. Cl. CPC A61L 9/12; A61L 9/042; A61L 9/048; A61L 2209/111; A61L 2209/13

(57) ABSTRACT

An apparatus, composition, and method for generating odor on demand. The apparatus includes a dispersing mechanism with a plurality of blister pockets each including a releasable aroma. A triggering mechanism is in combination with each of the plurality of blister pockets and configured to separately rupture individual blister pockets to release a corresponding releasable aroma. A controller is in actuating combination with the triggering mechanism. The controller is configured to monitor for at least one predetermined stimulus to actuate the triggering mechanism. The stimulus can include a predetermined text, sound, image, and/or location generated by the electronic device. A smart material can be used for holding and releasing the aroma or other compound. The smart material can include a porous polymer matrix, wherein the porous polymer matrix releases the material upon an actuation of the triggering mechanism. The invention further includes an electrospun fibrous structure with a hydrogel material having reversible properties for controlled release of the aroma, and a functionalized group to enhance water retention capability of the hydrogel in air. A physical property is reversibly altered as a result of a stimulus to release a compound or aroma and subsequently returns to an unaltered state resulting in containment of any remaining amounts of the compound or aroma.

### CROSS REFERENCE TO RELATED APPLICATION

This application claims the benefit of U.S. Provisional Patent Application, Ser. No. 63/405,182, filed on 9 Sep. 2022. The co-pending provisional application is hereby incorporated by reference herein in its entirety and is made a part hereof, including but not limited to those portions which specifically appear hereinafter.

### BACKGROUND OF THE INVENTION

This invention relates generally to providing aroma features to and/or via electronic devices, and more particularly to an apparatus and method for releasing/generating compounds or odors, which includes a dispersing mechanism including at least one releasable compound or aroma.

Consumer goods companies are heavily investing in virtual reality (VR) devices and/or digital assistant devices. Currently, text, images, and sound are transmittable electronically for these purposes. There is a continuing need or desire to improve the VR and digital marketplace.

### SUMMARY OF THE INVENTION

A general object of the invention is to provide smell to the attributes of electronic devices. The invention incorporates into the VR and digital experience by providing olfactory features to and/or via electronic devices.

The present invention includes an aromatizer device that can produce aroma based on a remote or local signal command. Embodiments of this invention include an aroma storage module, a stimulus, and a trigger mechanism.

Embodiments of this invention include an apparatus for generating odor, which includes a dispersing mechanism including at least one releasable compound, preferably an aroma, a triggering mechanism in combination with the dispersing mechanism and configured to trigger the dispersing mechanism to release the at least one releasable aroma, and a controller in actuating combination with the triggering mechanism. The controller is configured to monitor for at least one predetermined stimulus, such as a sound, image, and/or location, and to actuate the triggering mechanism upon determining an occurrence of the at least one predetermined stimulus. The dispersing mechanism desirably includes an aroma storage module, such as a polymer matrix and/or a blister pocket.

In embodiments of this invention, the compound/aroma storage module is a medium which securely stores aroma samples in desired conditions and will emit aromas only in response to a specific predetermined stimulant. One exemplary storage medium includes a polymer matrix with high retention capacity grafted with a smart polymer material that encapsulates the matrix, and is impervious at ambient temperatures. When the temperature rises above a particular moderate temperature threshold the smart material reversibly shrinks/swells to allow the aroma to escape and disperse by, for example, a micro fan. Further operation can be in two modes: (1) once the trigger is removed it returns to its original state; or (2) heat triggers the complete release of an aroma from a micro-container that is disposable. An array of aroma compounds can be printed on a disposable sheet, and a specific aroma can be release when a “pixel” is registered by a heat source.

The micro fan can have a bi-directional motor and two functions: a) in the forward direction it blows on the polymer to disperse the aroma and to cool down the polymer. After a predetermined time the fan reverses direction and acts as an exhaust fan to evacuate the chamber of aroma residues.

An exemplary smart material of this invention is or includes hydrogel, such as poly(N-isopropylacrylamide) (PNIPAAm). The polymer can be chemically cross-linked by acrylamide and N,N′-methylenebisacrylamide to improve the stability and mechanical strength. The transition temperature can also be modulated by the extent of cross-linking. Different members of the hydrogel group can be used with different response temperatures for various applications and conditions. The matrix can be a porous polymer such as cellulose, polystyrene, etc.

Embodiments include modified hydrogels for use, particularly modified PNIPAAm. Applicability of PNIPAAm to applications outside liquid media can be limited by loss of water in ambient air, resulting in inadequate response to stimulants. In embodiments of this invention, a modified smart material (e.g., PNIPAAm) is complexed with a metal-organic frameworks (MOF), such as via physical blending or functionalization to capture water from the air even at low relative humidity. Additionally or alternatively, the smart material incorporates compounds of cyclodextrin or similar materials which have a favorable encapsulation/slow release capacity for aroma, etc. Advanced fabrication methods such as electrospinning can additionally or alternatively be used in a highly controllable manner.

The invention further includes a smart material for holding and releasing a material, such as an aroma. The smart material includes a porous polymer matrix that releases the material upon an actuation of a triggering mechanism. In embodiments, the porous polymer matrix, preferably PNIPAAM, is cross-linked or mixed with hydrophilic components such as PEG, glycerin, sodium alginate, calcium chloride, zeolite, silica gel or silicone elastomer.

In embodiments of this invention, the porous polymer matrix, preferably PNIPAAM, is treated to reduce drying in ambient air. The porous polymer matrix, for example, can be combined with a metal-organic framework (MOF), such as iron-based MOF (MIL 100), zirconium-based MOF (801 or 841), aluminum-based MOF (303), and combinations thereof.

In embodiments of this invention, the porous polymer matrix is embodied as an electro-spun fiber. The porous polymer matrix can be encapsulated in cyclodextrin (alfa, beta, or gamma), such as through co-axial electrospinning, whereby a core comprises cyclodextrin and a PNIPAAm complex will form the shell.

In embodiments, the polymer matrix comprises microgel droplets, such as including nano-sized polymer matrix particles. The dispersing mechanism can include microgel droplets of polymer matrix particles having particle diameters of less than 500 nanometers, more preferably less than 400 nanometers, and desirably 250 nanometers or less. For example PNIPAAm microgel particles can be approximately 60 nanometers when ‘loaded’ with components according to this invention, and reduce to about 30 nanometers upon releasing the aroma compound.

In embodiments of this invention, the porous polymer matrix comprises additives such as carbon nanotubes (CNT), graphene, graphene oxide (GO), calcium chloride, silica gel, titanium oxide, and combinations thereof, such as to improve light/IR absorption/response or other characteristics of the polymer complex.

The invention further includes a polymer matrix for encapsulating a compound, such as an aroma material, a drug material, a cosmetic material, and/or a nutritional material, that can be released upon a stimulus, such as a thermal, optical, chemical, pH-based, electrical, magnetic, vibrational or mechanical stimulus. The polymer matrix includes: a hydrogel material having reversible properties (e.g., PNIPAAm) for controlled release; and a functionalized group (e.g., MOF) to enhance water retention capability of the hydrogel in air. The hydrogel material and the functionalized group can include or be embodied as a fibrous architecture formed by electrospinning, wherein at least one physical property of the polymer material is reversibly altered as a result of a stimulus to release the compound and subsequently returns to an unaltered state resulting in containment of any remaining amounts of the compound.

The polymer matrix can include an encapsulation compound (e.g., cyclodextrin) for slow release of the compound. The polymer matrix can additionally or alternatively include additives such as carbon nanotubes (CNT), graphene, graphene oxide (GO), calcium chloride, silica gel, and/or titanium oxide, to enhance the effect of the stimulus.

The invention further includes an apparatus including a dispersing mechanism including at least one releasable aroma and the smart material or polymer matrix disclosed herein. Again a triggering mechanism is used in combination with the dispersing mechanism and configured to trigger the dispersing mechanism to release the at least one releasable aroma. A controller is desirably in actuating combination with the triggering mechanism, wherein the controller is configured to monitor for at least one stimulus such as a predetermined text, sound, image, and/or location and to actuate the triggering mechanism upon determining an occurrence of the at least one predetermined text, sound, image, or location.

The invention further includes a smart polymer matrix for encapsulating a compound. The matrix includes: a smart hydrogel material having reversible properties (e.g., PNIPAAM) for controlled release; a functionalized group (e.g., MOF) to enhance water retention capability of the hydrogel in air; an encapsulation compound (e.g. cyclodextrin) for slow release of aroma; and a structural architecture formed by electrospinning the hydrogel and the functionalized group into fibers. At least one physical property of the smart material is reversibly altered as a result of a stimulus so that the smart material responsively reacts to the stimulus resulting in a controlled release of the compound and subsequently returns to an unaltered state resulting in containment of any remaining amounts of the compound.

Embodiments of this invention include smart materials/devices that can harvest aroma compounds and release them in a controllable way. Embodiments of this invention include smart materials and/or sensors that sense an undesirable compound as a stimulus and trigger the release of a desirable aromatic compound to mask/neutralize the undesirable compound.

In embodiments of this invention, the apparatus contains a sensor to transmit a signal upon sensing undesirable odors, such as, without limitations, waste, sewer, and other industrial, commercial or domestic undesirable odors. When the sensor detects the odor to exceed a threshold, the sensor actuates the triggering mechanism to release specific desirable (good) aroma compounds. The design can be in such a way that aroma compounds in multiple “micro containers” can be released subsequently to mask the odor. The emission of good odor will continue until the undesirable odor is completely masked and the odor sensor detects it at less than threshold level. Then, the stimulation is terminated.

The triggering mechanism is desirably selected based on the requirement of the aroma storage module. When the smart material reversibly changes its characteristics based on temperature, any heat generating device such as electrical wire or light can be used, such as a low-power IR laser source. To effectively absorb the IR light and quickly disperse the heat within the polymer, graphene oxide nanoparticles or carbon nanotubes can be incorporated into the PNIPAAm. In this way the response time can be improved significantly. Embodiments of this invention further include materials and/or coatings to preserve the absorption and sealing effect promoted by incorporation of, for example, graphene oxide (GO) to PNIPAAm, and/or to provide long-term water retention for PNIPAAm-GO.

The trigger mechanism is chosen based on the application. The trigger is generally powered electrically, and the command can be actuated in any form that is electrically addressable via, for example, Wi-Fi, Bluetooth, GPS, etc. Commands that are particularly applicable for VR devices include voice command, music recognition, and/or image recognition. As an example for a VR headset, the trigger mechanism can be actuated by push of a handle or touch of a button by a user. For web applications it can be actuated by a click of a mouse on a certain picture on a website, and for a digital assistant device, such as ALEXA, it can be a “wake-up word”.

With emergence of voice recognition and voice commands in emerging technologies, voice/sound is one of the trigger mechanisms that can be used in this invention. As an example, a digital assistant (e.g., ALEXA) for example, can play music from a playlist, if digital assistant or the aromatizer device receives a voice command to play a certain romantic song containing the word “rose”, the aromatizer can create aroma of rose whenever the word is heard by ALEXA in the song. Of course it all depends on the list of aromas encapsulated in the aromatizer inventory.

Using text and/or image detection, the aromatizer can be actuated when a particular word and/or image is shown on a digital display, such as a VR screen or digital picture frame. For example, an image of a birthday party can cause actuation of a chocolate (cake) smell. As another example, playing a virtual fire on a screen can actuate a campfire smell.

A device equipped with a GPS or other motion/location system can trigger a certain aroma at given GPS coordinates. An example application is aromatizer/GPS equipped glasses used by blind people to locate their coordinates or to warn them when they approach an intersection, etc.

VR glasses equipped with the aromatizer used as a video game showing a combat scene can disperse, for example, smoke aroma, when the user pushes or triggers a handle.

Equipping medical instrumentations for brain studies such as Mill with the aromatizer to release aroma and/or drug as a tool to study or treat certain brain or other organ deficiencies. As an example, an Alzheimer patient's brain can be studied when a perfume that he/she used to wear is dispersed vs. a new perfume.

The shape of the device depends on the application, ranging from small spots on a rectangular or circular board as shown in FIGS. 3-8 or concentric rings of aroma 12 on a circular substrate as shown in FIG. 2. Various applications, forms, and shapes of the invention are possible. The aroma can be oil-based, water-based, a single-component, and/or a multi-component mixture. The distribution media can be a disc, a mini-disc, a chip, and/or a cartridge. The storage-media can be a hollow-fiber, a sheet, a cavity (chip, etc.), and/or a planar sheet. The encapsulation-media can be a hydrogel with different response temperatures, a hydrogel-grafted on a high permeability polymer composite, an electrospun fiber, a polymer with active element (VLA-CNT, etc.), and/or small bubbles. The trigger can be heat (electrical), vibration (piezoelectric crystal, etc.), light (visual, laser, UV, etc.), magnetic, and/or a mechanical force.

The invention further includes an apparatus for generating odor, including a dispersing mechanism comprising a plurality of blister pockets each including a releasable aroma, a triggering mechanism in combination with each of the plurality of blister pockets and configured to separately rupture individual blister pockets to release a corresponding releasable aroma, and a controller in actuating combination with the triggering mechanism, wherein the controller is configured to monitor for at least one predetermined stimulus to actuate the triggering mechanism.

In embodiments of this invention, the plurality of miniscule pockets (blisters) are formed on a conductive segment of the substrate. Each conductive segment can be connected via two tiny wires (or printed board) to the controller or the power source, upon demand bursting (polymeric materials) or melting (beeswax, starch) the pockets by thermal energy. In some embodiments of this invention, the plurality of miniscule pockets (blisters) are formed on the substrate and a retractable piercing needle can rupture the dome of the miniscule by mechanical or thermo-mechanical force. In some embodiments of this invention, the plurality of miniscule pockets (blisters) are ruptured by optical or thermal energy.

Applications of this invention include, without limitation, ecommerce/online advertising, virtual/augmented reality, video games, intelligent voice interaction/personal assistant, robotics, personal ambience, marketing of products with aromas (e.g., food, wines or perfume), phone companions, and/or medical.

Other objects and advantages will be apparent to those skilled in the art from the following detailed description taken in conjunction with the appended claims and drawings.

### BRIEF DESCRIPTION OF THE DRAWINGS

FIG. 1 illustrates use of an apparatus according to one embodiment of this invention.

FIG. 2 illustrates an apparatus substrate layout according to one embodiment of this invention.

FIGS. 3-5 illustrate an apparatus according to one embodiment of this invention.

FIGS. 6-8 illustrate an apparatus according to one embodiment of this invention.

FIG. 9 is a chart summarizing materials according to embodiments of this invention.

### DESCRIPTION OF PREFERRED AND ILLUSTRATED EMBODIMENTS

The invention includes an apparatus for generating odor. The apparatus of embodiments of this invention generally includes a substrate, a dispersing material, and a device for determining a stimulus and providing a trigger to the dispersing material upon demand as a function of a signal.

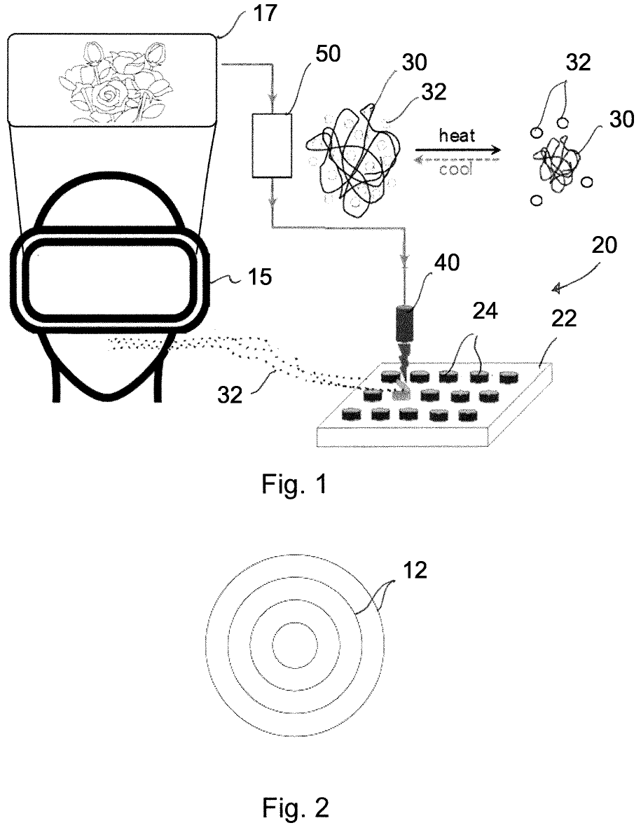

FIG. 1 generally illustrates an apparatus 20 according to one embodiment of this invention in combination with a VR headset 15. The apparatus 20 includes a dispensing mechanism embodied as a plurality of blisters 24 on substrate 22. Each blister 24 includes a releasable aroma, illustrated in FIG. 1 as a polymer matrix 30 that releases an aroma compound 32, such as upon being heated. A triggering mechanism, namely an infrared light or laser 40, is used to rupture the blister and/or heat the polymer matrix 30. A controller 50 is in actuating combination with the triggering mechanism 40, such that the controller 50 monitors for a predetermined stimulus from the VR headset 15 and actuates the triggering mechanism 40 upon determining an occurrence of the predetermined stimulus. In the illustrated embodiment, when the VR headset screen 17 shows flowers, the triggering mechanism 40 is directed to a blister containing compounds that create a flower aroma. The triggering mechanism 40 ruptures the corresponding blister 24 and the aroma 32 wafts to the wearer of the VR headset 15 to coordinate the olfactory experience with the visual/audio experience of the headset 15.

The invention can be actuated manually, such as pushing a button on a VR headset, but is desirably automated via monitoring and detecting a stimulus related to the aroma to be released. The apparatus of this invention can include a controller in actuating combination with an aroma triggering mechanism. The controller is configured to automatically monitor for at least one predetermined text, sound, image, and/or location as a stimulus, and to actuate the triggering mechanism upon determining an occurrence of the at least one predetermined text, sound, image, or location. The sound may be a word or other noise (e.g., wind, waves, roar, etc.) that is related to a contained aroma. For example, the word “rose”, such as when played through a music player, can actuate a rose aroma. Likewise detection of a photo of a rose on an electronic device display can actuate the rose aroma. The controller can be integrated into a device, such as a VR device or digital assistant device (ALEXA, SIRI, GOOGLE, etc.) or can be a separate device used in proximity.

The dispersing material can be nonporous and/or include a compound having non-reversible properties, and the dispersing material retains at least one releasable aroma positioned on the substrate. Desirably at least one mechanical/physical/chemical property of the dispersing material is non-reversibly altered as a result of the triggering so that the dispersing material responsively reacts to the triggering resulting in a release of aroma.

Exemplary dispersing materials include hydrogels with different response temperatures. Since the device will be used around the world at different ambient temperatures, smart materials (hydrogel) with different response (transition) temperatures may be needed to expand the applicability of the technology. Hydrogels include, for example, poly(N-isopropylacrylamide) (PNIPAAm) crosslinked by acrylamide and N,N′-methylenebisacrylamide with different extents of cross linking (transition temperature at −35° C.), or poly[2-(dimethylamino)ethyl methacrylate] (PDMAEMA) with a transition temperature of around 50° C. Another dispersing material includes electrospun fiber. Electrospinning produces nanometer diameter fibers. A coaxial spinneret design allows for the non-spinnable aroma compound to be encapsulated into polymer nanofibers. These nanofibers provide tremendous aroma storage capacity in a small volume. Once grafted with smart material such as PNIPAAM, etc., the number and/or amount of aromas can be increased in a small volume. Also, if the response time of a bulk hydrogel is considered slow, forming a nanofibrous mesh from electrospun fibers provides high porosity and surface area, and the structure is able to respond at a much faster rate than a bulk gel.

Exemplary dispersing materials further include polymers with active elements (VLA-CNT, etc.). Response time of the smart material can curtail the operation of the aromatizer. Improvement has been achieved by functionalizing or incorporating relevant compounds such as CNT (carbon nanotube) or graphene/graphene oxide nanoparticles, etc. These compounds can effectively improve the visual or infrared light absorption capacity and thermal distribution of the polymer matrix. Polymers with active elements for optical/chemical triggering can also be used. Titanium oxide (TiO2) is an exemplary photoactive element that can be incorporated in the polymer matrix; the anatase form of the oxide has photo catalytic activity under UV light. Addition of such oxide is envisioned for applications where optical/chemical triggering is employed. In addition, amphiphilic interpenetrating polymer networks (IPN) or semi-interpenetrating polymer network (SIPN) polymeric composites can be used, and are obtained by interpenetration of a linear or branched polymer within a network of another crosslinked polymer. Amphiphilic SIPNs are produced when one of the polymeric components is hydrophobic and the other is hydrophilic. This also can be prepared on a matrix of hydrophobic polymeric or elastomeric porous particles, such as recycled tire particles which contain porous carbon black. Amphiphilic SIPNs retain water based odors and will release them upon heating or exposure to air by removing its cover.

Embodiments of this invention are adapted to preserve the IR absorption and/or sealing effect promoted by incorporation of oxides, such as graphene oxide (GO), to PNIPAAm, and/or to provide long-term fluid retention for PNIPAAm-GO. In one exemplary embodiment, a rubbery material, such as an elastomer (e.g., silicone rubber), is wrapped or otherwise coated around the smart material (PNIPAAm-GO) to keep the hydrogel material from drying. The thin wrapping material can be coated/treated, such as dipped into a solution of benzophenone, before application. In another exemplary embodiment, the smart material is coated with a silicone compound, e.g., MasterSil™ 151, that is a medical grade optically clear, low viscosity silicone product. Additionally or alternatively, hygroscopic materials such as PEG and/or glycerin can be added to a reservoir for improved water retention capability. PEG-based hydrogel materials such as PEG-diacrylate (PEGDA) can also be incorporated for improving storage capability. Incorporating hydrophilic co-polymers to PNIPAAm chains can also be used for improved water retention/reversibility capability.

Embodiments of this invention further include smart materials (e.g., a hydrogel) with different response (transition) temperatures to improve performance and to expand applicability. One example is poly[2-(dimethylamino)ethyl methacrylate] (PDMAEMA) with a transition temperature of around 50° C. In addition, dual stimulus sensitive thermo- and/or pH-responsive grafted hydrogels of poly(N,N-dimethylaminoethyl methacrylate) (PDMAEMA) and poly(N-isopropylacrylamide) (PNIPAM) can be used for rapid response and high swelling ratio.

Embodiments of this invention include an apparatus for generating odor, the apparatus including a substrate and a non-porous material having a plurality of miniscule pockets (blisters) retaining at least one releasable aroma positioned on the substrate. The material can be a polymer having non-reversible physical properties.

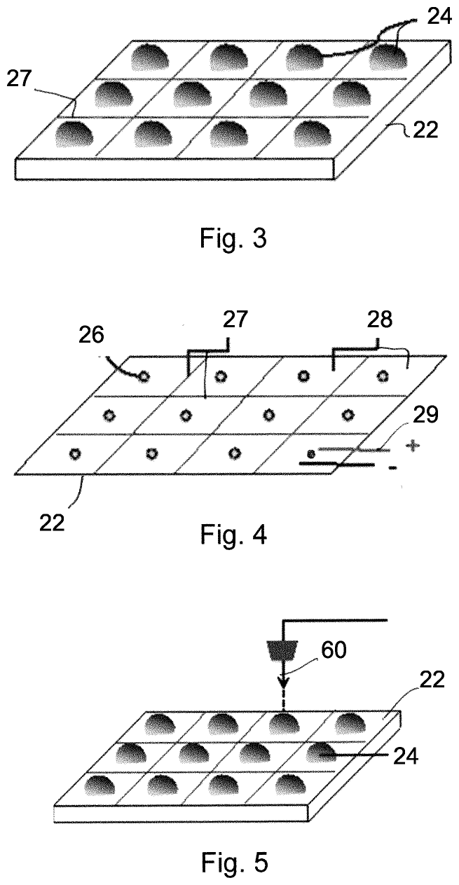

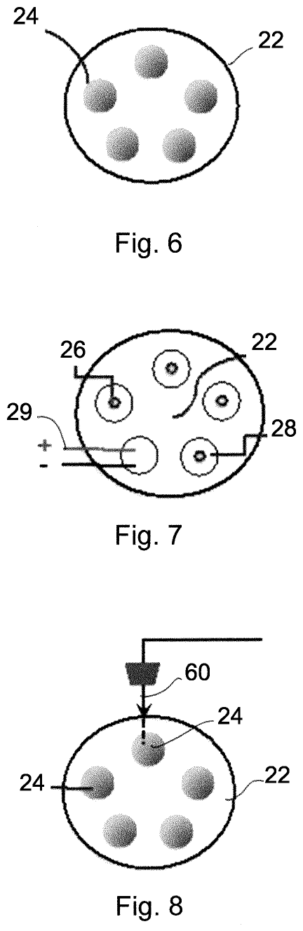

FIGS. 3-8 show dispersing mechanism embodiments which include a substrate 22 with a segmented matrix of miniscule thin non-smart polymer bubbles (blisters) 24 on top. Each bubble or row of bubbles 24 can contain a specific/different aroma. The polymer material is desirably non-porous and non-smart. In some embodiments, the substrate 22 includes conductive segments 28 separated from each other by non-conductive materials 27. In some embodiments of this invention, shown in FIG. 4, the underneath of each miniscule pocket (blister) includes a tiny hole 26 which will be sealed after injecting the aroma to the pocket by a syringe. Furthermore, as shown in FIG. 4, each conductive section 28 is connected with wires 29 to a controller and power source. Upon demand the appropriate conductive segment is heated by power through the wires 28 and any suitable switches, bursting the appropriate bubble 24 and releasing the aroma due to softening of internal polymer matrix or internal pressure build-up of aroma. FIGS. 6 and 7 show a similar structure, except in a circular configuration.

As illustrated in FIGS. 5 and 8, a retractable electromechanical needle 60 is used to rupture the bubble 24 to release the aroma. Still in another embodiment, the laser source can melt the bubble to rupture it or it can build pressure inside the bubble by raise the temperature of the aroma inside the bubble and rupturing it. Furthermore yet in another embodiment, the odor may be encapsulated in the form of gas or paste in very small bubbles with thin skin that can ruptured upon mechanical force. For example, by pushing a mesh cover with holes less than bubble diameters to release the odor.

Exemplary non-porous materials include polymers (mechanical trigger: MYLAR, TYGON, rubber, etc.), transparent polymers (optical trigger: silicone rubber, etc.), and/or natural compounds (thermal trigger: beeswax, starch, etc.). The substrate is desirably a non-conductive material (polymer) or conductive (copper, aluminum, etc.) segments separated by non-conductive materials, or combinations thereof. The trigger can be a thermal trigger and provided by a laser, such as shown in FIG. 1, or an IR or UV source. The trigger can be mechanical, such as the piercing needle of FIGS. 5 and 8. The trigger can further be optical, electrical, electromagnetic (heated or non-heated), piezoelectric (vibration), or chemical.

Embodiments of this invention include a smart material that can release a desirable compound when it is exposed to a particular undesirable compound (e.g., sulfur compound-hydrogen sulfide). In one example, the material is in a shape of a hollow fiber tube filled with an aroma compound and closed or otherwise sealed by a membrane. The fibers can be any suitable shape (e.g., cylindrical or non-cylindrical) or formed with any suitable method (e.g., electrospun fibers) that provides excessive capacity in a limited space.

In one embodiment, once the smart material is exposed to the undesirable compound, pores of the smart material open reversibly due to a change of pH of the smart material because of exposure to an acidic sulfur compound, releasing the desirable compound contains therein. Once the exposure to the sulfur compound is terminated, the pores return to the original ‘closed’ position. In another embodiment, the pores of the smart material open up due to temperature change as a result of heat from reaction with or adsorption of the undesirable compound (e.g. sulfur compound), releasing the desirable compound. Once the exposure to the sulfur compound is terminated, the pores return to the original close position. Exemplary pH sensitive smart materials include ionic hydrogels. Cationic hydrogels such as chitosan and poly(ethylene imine) swell at low pH (acidic medium). Anionic hydrogels like carboxymethyl chitosan swell at higher pH (basic medium) due to ionization of the acidic groups.

In another embodiment of the invention, the material is a non-smart hollow fiber filled with a suitable adsorbent (e.g., the adsorbent can be activated carbon, silica, GC packing material, etc.) that is saturated with a desirable aroma and placed in a container equipped with a sensor and a heater. Once the sensor detects a predetermined undesirable compound, the heater is activated. As a result of increasing the temperature inside the container, the adsorbent material releases (desorbs) the desirable aroma compound. Once the concentration of the undesirable compounds drops below certain limit, the heater turns off, stopping emission of the aroma compound.

In yet another embodiment, the material is a non-smart hollow fiber filled with a suitable adsorbent that is saturated with a desirable aroma and placed in a container. The adsorbent has an affinity for an undesirable compound, such that once the undesirable compound (e.g. sulfur compound) makes contact, the adsorbent releases the desirable compound to make room for adsorbing the undesirable compound. Once the concentration of the undesirable compound drops below a certain limit, emission of the aroma compound stops.

In yet another embodiment, the material is a smart or non-smart hollow fiber filled with suitable encapsulated adsorbent that is saturated with a desirable aroma or an encapsulated liquid aroma compound within a container. An undesirable compound reacts with the encapsulation material to puncture the encapsulation shell, and releasing the desirable aroma compound. Optionally any liquid material vaporizes from the punctured shell.

In an alternative embodiment the apparatus includes a sensor that detects an undesirable compound and triggers release of a desired aroma.

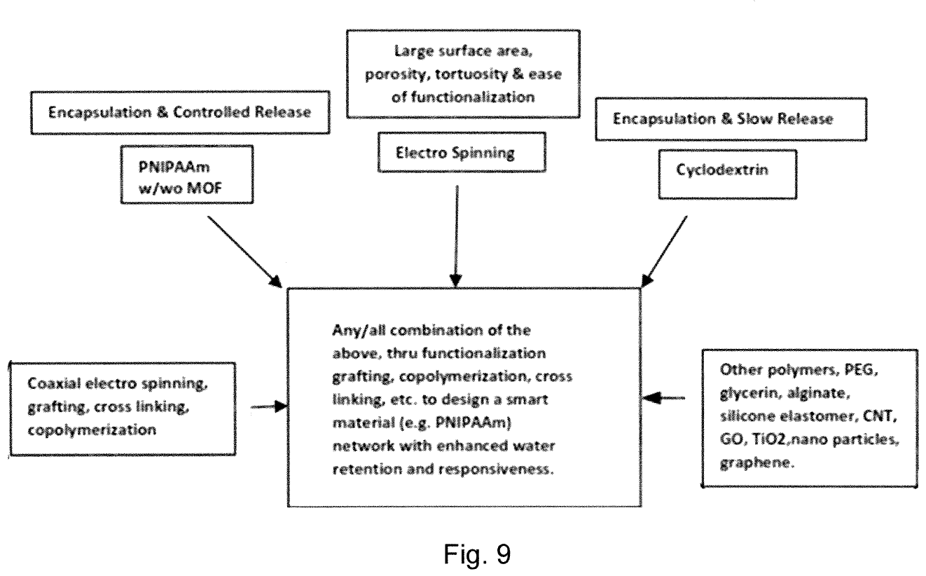

Embodiments of this invention, such as summarized in FIG. 9, incorporate approaches, or a synergy of several approaches, to improve the stimuli-responsive hydrogels for use, particularly PNIPAAm. Applicability of PNIPAAm to applications outside liquid media can be limited by loss of water in ambient air, resulting in inadequate response to stimulants. In some embodiments, this problem is overcome by functionalizing the smart materials to capture water from the air at ambient conditions. One approach includes a modified smart material (e.g., PNIPAAm) by addition of complex metal-organic frameworks (MOF), such as via physical blending or functionalization to capture water from the air even at low relative humidity. In another example embodiment, the invention additionally or alternatively incorporates compounds of “cyclodextrin” which have a favorable encapsulation/slow release capacity for aroma, etc. Advanced fabrication methods such as electrospinning can additionally or alternatively be used in a highly controllable manner. These above approaches can be used individually or collectively to provide smart materials with enhanced applicability. These approaches can also be used to expand the usefulness of the base smart materials of this invention, such as PNIPAAm, for example, without limitation for use in drug delivery, food, sensors, batteries, etc.

In embodiments of this invention, metal-organic frameworks (MOFs) are used to reduce or eliminate moisture loss from the smart materials. MOFs useful in this invention include compounds composed of metal (Zr, Zn, Al, Fe, V, Cr, Ni, etc.) ions or clusters linked by organic ligands (e.g., carboxylate). These hybrid organic and inorganic materials not only combine the respective benefits of organic and inorganic components but also often exhibit synergistic properties that exceed what would have been expected from the mixture of the two. MOFs provide a unique class of micro or mesa porous materials capable of trapping water at relative humidity levels as low as 10% and releasing them with ease at higher temperatures. Exemplary materials include, without limitation, Fe-based MOF MTh 100, Al-based MOF 303, Zr-based MOF 801 and/or MOF 841, and combinations thereof. Each is capable of adsorbing moisture from air at relative humidity levels as low as 20% at 25° C. and releasing it if heated at higher temperatures (e.g., 45° C.). MOFs have unique properties ranging from high surface area, in some MOFs the area can be over 8000 m2/gr to the recently discovered property of being able to conduct charge (e.g., Ni-based MOF) by various mechanisms.

In embodiments of this invention, cyclodextrins are used for controlling the rate of release, and/or to assist in reducing or eliminating moisture loss from the smart materials. Cyclodextrins are a family of cyclic saccharides. Typical cyclodextrins contain a number of glucose monomers ranging from six to eight units in a ring, creating a cone shape: alpha-cyclodextrin has 6, beta has 7 and gamma has 8 glucose units. Cyclodextrins are generally white, water-soluble solids with minimal toxicity. Cyclodextrins tend to bind other molecules in their quasi-cylindrical interiors. The low toxicity and molecular structure make them ideal for food, cosmetic, drug and flavor encapsulation and slow release.

In embodiments of this invention, electrospinning is used to prepare a smart material structure with very high porosity and enhanced capabilities, and/or to assist in reducing or eliminating moisture loss from the smart materials. Electrospinning is a simple, low-cost and highly efficient technique to generate desirable nano/micro-fibers from polymer solutions. The basic electrospinning contains a high-voltage system, spinneret, and collector. Nano/micro-fibers are drawn from a solution droplet at the tip of a needle due to the applied high voltage between the tip and the grounded fiber collector. Under the influence of the electric field, a liquid jet is emitted. As the jet travels in the air, the solvent evaporates, leaving polymer fibers landing on the grounded collector. Additional components, such as MOF, can be easily incorporated in a polymer solution to generate composite fibers with added functionality and features.

Embodiments of this invention include MOF-PNIPAAm composite fibers, wherein MOF's water sorption property allows water retention around PNIPAAm during its storage, to overcome expected dehydration. When stimulated by IR beam, MOF-PNIPAAm fibers shrink to release the encapsulated compounds while the moisture is removed to achieve MOF recovery. The recovered MOF is capable of re-capturing water from the ambient air to rehydrate PNIPAAm for future usage. This design allows repeated release and encapsulation of aroma (or other compounds) in a highly controllable fashion. PNIPAAm-cellulose fibers can be generated similarly. Electrospinning is a versatile and scalable fabrication technique to generate fibers with highly controllable fiber architecture, size, tortuosity, porosity and degradation rate. Coaxial spinning is an alternative method of co-spinning to produce composite fibers. In this case, the aroma compound forms the core while the MOF-PNIPAAm contributes to the shell in the fibers to achieve aroma encapsulation and release.

Embodiments of this invention include microgel droplets, such as including nano-sized polymer matrix particles. The particular nanosize of the polymer matrix particles can vary, depending on need and/or components used. For example, the polymer matrix particle size can be about 20 nm to about 300 nm. The matrix particles generally have a larger size when loaded with compounds (e.g., aroma) and/or components (e.g., MOFs) of this invention, and shrink to a smaller size when releasing the aroma or other compounds. In embodiments, PNIPAM microgel nanoparticles are about 30-100 nm in diameter, and preferably about 60 nm in diameter. These respond to photothermal stimulation and reduce approximately 50% (e.g., 60 to 30 nm) upon releasing the encapsulated aroma compound. As another example, synthesized PNIPAM-beta cyclodextrin microgel nanoparticles were approximately 200 nm in diameter, and reduced to about 90 nm upon photothermal stimulation to release the encapsulated aroma compound. PNIPAM-beta cyclodextrin nanoparticles can thus load aroma compound more efficiently. MOF 303 particles have varied sizes around 1 micrometer. PNIPAM-MOF particle dimension is largely determined by MOF particle size.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### Examples

### 1. MOF-PNIPAM Microgels

MOF-PNIPAM microgels were synthesized by opting for a 50:5.5 mole ratio of NIPAM and MOF-303(Al) (Alfa chemistry, NY, USA), in combining with the cross-linker MBA, SDS surfactant, and KPS initiator. The successful synthesis of microgels with a hydrodynamic diameter of 340±40 nm was validated through Fourier-transform infrared spectroscopy (FTIR) and differential scanning calorimetry (DSC) analyses. Notably, these microgels exhibit thermo-responsive behavior that is both reversible and manifests a volume phase transition temperature (VPTT) of approximately 45° C., as determined by dynamic light scattering DLS analysis.

Quantitative evaluation revealed the microgels' capacity to encapsulate approximately 17.5% of thymol from an initial 6 mg of thymol, utilizing 180 mg of microgels. Furthermore, the moisture-absorption capability of these microgels was evident, with 1 mg of microgel able to capture 1.9 mg of water when exposed to a moisture environment at −20 in Hg and 25° C. for a duration of 24 hours. Based on quantitative approach, 4.8% of thymol was released upon 60 seconds of laser irradiation from pre-wet 3 mg thymol-encapsulated microgels. MOF-303(Al) was introduced into the microgels because of its exceptional water harvesting capability from ambient air and other impressive properties including high surface area, pore size, thermal stability and encapsulation ability.

Incorporation of MOF to the MOF-PNIPAM microgels eliminated the need for addition of 10 μl of water (as opposed to the case with the β-cyclodextrin-PNIPAM without MOF, described in the next section) needed for release of thymol. The amount of thymol encapsulation was lower compared to the case with β-cyclodextrin-PNIPAM. Because of that our next step is using a combined complex (β-cyclodextrin-PNIPAM-MOF) to capitalize on moisture retention capability of MOF and better encapsulation/controlled release ability of β-cyclodextrin. Moreover, MOF-303(Al) can be used efficiently for water capture and release due to their stability and highly hydrophilic nature.

### 2. β-Cyclodextrin-PNIPAM Microgels

To synthesize microgel nanoparticles of β-CD-PNIPAM, a precipitation emulsion polymerization method was employed in an aqueous environment. In this approach, microgels were fabricated from 106 mmol of N-isopropylacrylamide (NIPAM) and beta-Cyclodextrin (β-CD) at a mole ratio of 1:0.06, along with 5.3 mmol of the crosslinker N, N′-methylenebisacrylamide (MBA). This reaction also involved the use of sodium dodecyl sulfate (SDS) as a surfactant and potassium persulfate (KPS) as an initiator. The successful synthesis of microgels was confirmed through Fourier-transform infrared spectroscopy (FTIR) and differential scanning calorimetry (DSC).

These microgels exhibited a hydrodynamic diameter of approximately 209±25 nm, as determined by dynamic light scattering (DLS). Scanning electron microscopy (SEM) analysis revealed a particle size of around 96±6 nm in dry form at 25° C. The microgels exhibited a volume phase transition temperature (VPTT) of approximately 33° C., as indicated by both DLS and DSC. Thymol is an aromatic compound and tonic, extracted from plants like Thymus, Satureja, Euphrasia Rostkoviana etc. Thymol purifies she air, facilitates respiration, and enhances the well-being of the body and mind. Thymol, when encapsulated, could be loaded into these microgels up to 82.6% of its weight (6 mg of thymol with 720 mg of microgels).

Photothermal release of thymol from microgels was carried out using an 808 nm laser (DC12V, China). Quantitative analysis of thymol release demonstrated that a 20-second laser exposure, following the addition of 10 μl of water, resulted in the release of 10.6% of encapsulated thymol from 3 mg of thymol-loaded microgels. Qualitatively, a brief 20-second laser treatment of thymol-loaded microgels led to a notable response, with 86.6% of participants reporting the distinct scent of thymol.

Thus the invention provides an apparatus and method for releasing aroma, which can be paired with electronic devices to match the aromas to images or words on the devices.

The invention illustratively disclosed herein suitably may be practiced in the absence of any element, part, step, component, or ingredient which is not specifically disclosed herein.

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

What is claimed is:

1. An apparatus for generating odor, the apparatus comprising:
a dispersing mechanism including a releasable aroma;
a triggering mechanism in combination with the dispersing mechanism and configured to trigger the dispersing mechanism to release the releasable aroma; and
a controller in actuating combination with the triggering mechanism, wherein the controller is configured to monitor for a predetermined stimulus and to actuate the triggering mechanism upon determining an occurrence of the predetermined stimulus.

2. The apparatus of claim 1, wherein the dispersing mechanism comprises an aroma storage module selected from a polymer matrix and/or a blister pocket.

3. The apparatus of claim 2, wherein the polymer matrix comprises a porous polymer substrate, a hydrogel, and/or a hydrogel with carbon nanotubes (CNT), graphene, and/or titanium oxide.

4. The apparatus of claim 2, wherein the blister pocket comprises a film enclosing a releasable aroma, wherein the triggering mechanism is configured to ruptures the film to release the releasable aroma.

5. The apparatus of claim 2, wherein the blister pocket encloses the polymer matrix.

6. The apparatus of claim 1, wherein the triggering mechanism comprises a mechanical trigger, an optical trigger, an electrical trigger, and/or a thermal trigger, the controller is integrated with an electrical device that produces or receives the at least one predetermined text, sound, image, and/or location.

7. The apparatus of claim 6, wherein the electrical device comprises a virtual reality device, a computer system, a GPS device, or a digital assistant device.

8. The apparatus of claim 7, wherein the controller monitors for a displayed image on or a spoken word to or from the electrical device.

9. The apparatus of claim 1, further comprising a hollow fiber tube and membrane, filled with an aroma compound, wherein upon exposure to an undesirable compound the membrane releases the aroma.

10. The apparatus of claim 1, further comprising a pH sensitive hydrogel including chitosan and poly(ethylene imine) or carboxymethyl chitosan.

11. The apparatus of claim 1, wherein the dispersing mechanism comprises a plurality of blister pockets each including a releasable aroma, and the triggering mechanism is configured to separately rupture individual blister pockets to release a corresponding releasable aroma.

12. The apparatus of claim 11, wherein the plurality of blister pockets is disposed on a substrate, and the triggering mechanism comprises: a retractable needle; a retractable laser; and/or a pair of conductive wires along the substrate and in combination with each of the plurality of blister pockets.

13. The apparatus of claim 11, wherein the substrate comprises a filling hole under each of the plurality of blister pockets.

14. The apparatus of claim 1, wherein the dispersing mechanism comprises microgel droplets including polymer matrix particles having particle diameters of less than 500 nanometers.

15. The apparatus of claim 1, wherein the dispersing mechanism comprises a smart material for holding and releasing the aroma, the smart material comprising a porous polymer matrix, wherein the porous polymer matrix releases the material upon an actuation of the triggering mechanism.

16. The apparatus of claim 15, wherein the porous polymer matrix is cross-linked or mixed with hydrophilic components selected from the group consisting of PEG, glycerin, sodium alginate, calcium chloride, zeolite, silica gel, silicone elastomer, or combination thereof.

17. The apparatus of claim 15, wherein the porous polymer matrix comprises PNIPAAM treated or functionalized to reduce drying in ambient air.

18. The apparatus of claim 15, wherein the porous polymer matrix is combined with a metal organic framework (MOF) selected from Fe-based MOF MIL 100, Zr-based MOF 801, Zr-based MOF 841, Aluminum-based MOF 303, or combinations thereof.

19. The apparatus of claim 15, wherein the porous polymer matrix comprises an electro-spun fiber.

20. The apparatus of claim 15, wherein the porous polymer matrix comprises an additives selected from carbon nanotubes (CNT), graphene, graphene oxide (GO), calcium chloride, silica gel, titanium oxide, and combinations thereof.

21. The apparatus of claim 15, wherein the porous polymer matrix is encapsulated in cyclodextrin.

22. The apparatus of claim 21, wherein the porous polymer matrix is electrospun with the cyclodextrin, whereby a core comprises cyclodextrin and a PNIPAAm complex will form the shell.

23. The apparatus of claim 1, wherein the dispersing mechanism comprises an electrospun fibrous structure including:
a hydrogel material having reversible properties for controlled release of the aroma;
a functionalized group to enhance water retention capability of the hydrogel in air;
wherein at least one physical property of the dispersing mechanism is reversibly altered as a result of a stimulus to release the compound and subsequently returns to an unaltered state resulting in containment of any remaining amounts of the compound.

24. The apparatus of claim 23, further comprising an encapsulation compound for controlled release of the aroma.
